(19) United States
(12) Patent Application Publication
(10) Pub. No.: US 2021/0128457 A1
(43) Pub. Date: May 6, 2021

(54) METHOD OF DELIVERING DRUGS TO INNER EAR FACILITATED BY MICROBUBBLES

(71) Applicants: National Defense Medical Center, TAIPEI (TW); National Taiwan University of Science and Technology, TAIPEI CITY (TW)

(72) Inventors: Ai-Ho LIAO, Taipei City (TW); Chih-Hung WANG, New Taipei City (TW); Cheng-Ping SHIH, Taipei City (TW)

(21) Appl. No.: 16/876,109

(22) Filed: May 17, 2020

(30) Foreign Application Priority Data
Nov. 5, 2019 (TW) 108140149

(51) Int. Cl. A61K 9/00; A61K 41/00; A61K 47/69; A61K 9/06; A61K 38/38; A61K 49/22

(52) U.S. Cl. CPC A61K 9/0046; A61K 41/0047; A61K 49/223; A61K 9/06; A61K 38/38; A61K 47/6925

(57) ABSTRACT

A method of delivering drugs to inner ear facilitated by microbubbles, including mixing a microbubble composition and a drug into a microbubble-drug mixture, applying the microbubble-drug mixture to middle ear cavity, and placing a mechanical oscillation wave source to ear canal or cranium located behind the ear. The mechanical waves generated by the mechanical oscillation wave source penetrate through tympanum or cranium, and induce the cavitation on the microbubbles in the middle ear cavity. Thus, the permeability of the round window membrane is increased, so that the drug penetrates into inner through the round window membrane. Therefore, the mechanical oscillation wave source induces the cavitation on the microbubbles in a non-invasive way.

### CROSS-REFERENCE TO RELATED APPLICATION

This application claims priority to Taiwan Application Serial Number 108140149, filed on Nov. 5, 2019, which is herein incorporated by reference in its entirety.

### BACKGROUND

### Field of Invention

The present disclosure relates to a method of delivering drugs to inner ear. More particularly, the present disclosure relates to a method of delivering drugs to inner ear facilitated by microbubbles.

### Description of Related Art

The clinical administration of drugs to the inner ear can be performed systemically or locally. Systemic treatment administrated by oral or intravenous allows drugs to act at the inner ear through blood circulation. Because the systemic administration has greater side effects and the inner ear has a blood labyrinth barrier, drugs have poor penetration. Thus, many studies have been focused on the local therapy in recent years, such as administrating drugs into middle ear cavity, so that drugs can be delivered into the inner ear by penetrating the round window membrane. Intratympanic injection of drugs can avoid side effects caused by systemic administration, and is also the most popular method in clinical use for delivering drugs locally into the inner ear. However, opening of the eustachian tube in the middle ear cavity facilitates drugs elimination, and round window membrane has low permeability to drugs, so the intratympanic injection of drugs into the inner ear is not efficient.

As a result, how to facilitate the efficiency of drug delivery into the inner ear through round window membrane, the existing technology needs to be improved.

### SUMMARY

The present disclosure provides a method of delivering drugs to inner ear, so that the effect of the drug located in the middle ear cavity penetrating into inner ear through the round window membrane can be achieved.

The present disclosure provides a method of delivering drugs to inner ear facilitated by microbubbles, comprising: providing a microbubble composition, wherein the microbubble composition comprises at least one first medium and a plurality of microbubbles dispersed in the first medium; providing a drug; mixing the microbubble composition with the drug to form a microbubble-drug mixture; applying the microbubble-drug mixture to the middle ear cavity; and applying a mechanical oscillation wave source to be non-invasive and indirect contact with the microbubble-drug mixture, wherein mechanical waves are generated by the mechanical oscillation wave source, the microbubbles of the microbubble-drug mixture in the middle ear producing a cavitation is induced by the mechanical waves, so as to increase the permeability of the round window membrane, thereby allowing the drug in the microbubble-drug mixture to penetrate the round window membrane into the inner ear.

In some embodiment, after the step of applying the microbubble-drug mixture to the middle ear cavity, the method further comprises filling a second medium into an ear canal; wherein the step of applying a mechanical oscillation wave source to be non-invasive and indirect contact with the microbubble-drug mixture, the mechanical oscillation wave source is in contact with the second medium in the ear canal, and the mechanical waves penetrate the eardrum and the microbubbles in the microbubble-drug mixture located in the middle ear are induced to produce the cavitation.

In some embodiment, the second medium comprises saline, a gel, or a combination thereof.

In some embodiment, the step of applying the mechanical oscillation wave source to be non-invasive and indirect contact with the microbubble-drug mixture, the mechanical oscillation wave source placed at a skull near an ear shell generates mechanical waves without chiseling the skull, and the mechanical waves penetrate the skull and the microbubbles in the microbubble-drug mixture located in the middle ear are induced to produce the cavitation.

In some embodiment, the cavitation is a stable cavitation or an inertial cavitation.

In some embodiment, the step of applying the microbubble-drug mixture to the middle ear cavity is to apply the microbubble-drug mixture to a round window membrane toward a middle ear cavity.

In some embodiment, the first medium comprises saline, a gel, or a combination thereof.

In some embodiment, the material of the microbubbles comprises albumin, a lysozyme, a polymer, a liposome or a combination thereof.

In some embodiment, the microbubbles have a particle size from 0.5 μm to 2.5 μm.

In some embodiment, the mechanical oscillation wave source comprises an ultrasonic device, a laser device, or a combination thereof.

In some embodiment, the concentration of the microbubbles in the microbubble-drug mixture ranges from 1×10⁶ to 2×10⁸ particles/mL.

### BRIEF DESCRIPTION OF THE DRAWINGS

The present disclosure can be more fully understood by reading the following detailed description of the embodiment, with reference made to the accompanying drawings as follows:

FIG. 1 is a bar chart illustrating result of ultrasound-induced microbubble cavitation caused by direct administration or transcranial administration of 1× or 10× dilution of the microbubbles according to one embodiment of the present disclosure.

FIG. 2 is a bar chart illustrating image intensity (decibel, dB) quantification of destruction efficiency before and after direct administration of 1 watt per square centimeter (W/cm²), 2 W/cm², 3 W/cm², and 4 W/cm² ultrasound according to one embodiment of the present disclosure.

FIG. 3 is a bar chart illustrating image intensity (dB) quantification of destruction efficiency before and after transcranial administration of 1 W/cm², 2 W/cm², 3 W/cm², and 4 W/cm² ultrasound according to one embodiment of the present disclosure.

FIG. 4 is a bar chart illustrating image intensity (dB) quantification of destruction efficiency before and after different approaches administration of 3 W/cm² ultrasound according to one embodiment of the present disclosure.

FIG. 5A is a cross-sectional view illustrating a single-membrane model for drug delivering; FIG. 5B is a schematic view of ultrasound probe perpendicular to the membrane of the single-membrane model for drug delivering according to one embodiment of the present disclosure; FIG. 5C is a schematic view of ultrasound probe deviated 20° from the center in the single-membrane model for drug delivering according to one embodiment of the present disclosure; FIG. 5D is a schematic view of the single-membrane model for drug delivering with transcranial administration according to one embodiment of the present disclosure; FIG. 5E is a cross-sectional view illustrating a two-membranes model for drug delivering according to one embodiment of the present disclosure; FIG. 5F is a schematic view of the two-membranes model for drug delivering according to one embodiment of the present disclosure.

FIG. 6 is a bar chart illustrating quantification of the experiment result of the models for in vitro drug delivering according to one embodiment of the present disclosure.

FIG. 7 is a schematic diagram of transcanal ultrasound-induced microbubble cavitation according to one embodiment of the present disclosure.

FIG. 8 is a bar chart illustrating quantification of the experiment result of transcanal and transcranial ultrasound-induced microbubble cavitation according to one embodiment of the present disclosure.

FIG. 9 is a bar chart illustrating quantification of click stimuli of auditory brainstem responses (ABRs) result after transcanal ultrasound-induced microbubble cavitation according to one embodiment of the present disclosure.

FIG. 10 is a bar chart illustrating quantification of different tone-burst stimuli of auditory brainstem responses result after transcanal ultrasound-induced microbubble cavitation according to one embodiment of the present disclosure.

FIG. 11 is a immunohistochemistry diagram of inner hair cells at basal, second, and third turns after round-window soaking (RWS) or transcanal ultrasound-induced microbubble cavitation (USM) in vivo according to one embodiment of the present disclosure; scale bar=50 μm.

FIG. 12 is a immunohistochemistry diagram of inner and outer hair cells at basal, second, and third turns after round-window soaking (RWS) or transcanal ultrasound-induced microbubble cavitation (USM) in vivo according to one embodiment of the present disclosure; green fluorescence indicates gentamicin, red fluorescence indicates actin, and blue fluorescence indicates cell nuclei; scale bar=50 μm.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

As used herein, the term “ear shell” is commonly known as ear having cartilage, and the cartilage maintains the shape of the ear.

As used herein, the term “skull near an ear shell” indicates that the skin is located outside the skull around the ear shell. The mechanical oscillation wave source is placed without removing the skull, such as in one embodiment, the ultrasound probe is directly attached on the skin outside the skull around the ear.

As used herein, the term “non-invasive” indicates that the device putting into the body does not involve, such as the device is located at the surface of the body, or the device is located in the ear canal but not penetrates through the eardrum. Non-invasive does not involve puncture or perform an operation etc.

As used herein, the term “cavitation” indicates that ultrasonic waves of a certain energy and audio are applied to the microbubbles, which will induce the cavitation. Cavitation can be divided in to two types, one is called stable cavitation, also called non-inertial cavitation: when the microbubbles are repeatedly compression and rarefaction under the action of the low acoustic energy of the ultrasonic wave, the liquid around the microbubbles will flow, so the drug delivery can be promoted. The other is called inertial cavitation: when the microbubbles are extremely compression and rarefaction under the action of the strong acoustic energy of the ultrasound, so that the pulse wave and the liquid jet are generated by the final collapse of the microbubbles, and these forces can enhance the drug absorption at the target site.

In some embodiments of the present disclosure, the drugs include, but are not limited to, small molecule drugs and large molecule drugs. Small-molecule drugs refer to drugs made of chemical synthesis; large-molecule drugs refer to drugs made of biotechnology (for example, microorganisms, plants, and animal cells etc.).

The microbubble ultrasound contrast agent of some embodiments of the present disclosure may be in the form of an aqueous solution or a gel form. The material of the microbubbles can be roughly divided into three categories: albumin microbubbles, lysozyme microbubbles, liposome microbubbles or polymer microbubbles. The microbubbles contained in the microbubble ultrasound contrast agent have stable shells and may be used to enhance the scattering signals of reflected ultrasound. Under various ultrasound energy intensities, using the microbubble ultrasound contrast agent can increase the penetration depth (i.e. absorption efficiency) and/or the amount of penetration (i.e. absorption) of the chemicals or small molecules at the applied area.

In some embodiment, the microbubble composition as used herein is also called the microbubble ultrasound contrast agent.

In some embodiments, the concentration of microbubble-drug mixture ranges from 1×10⁶ to 2×10⁸ particles/mL. In one embodiment, the concentration ranges from 2×10⁶ to 2×10⁸ particles/mL. In one embodiment, the concentration ranges from 2×10⁷ to 2×10⁸ particles/mL. In one embodiment, the concentration ranges from 1×10⁶, 2×10⁶, 3×10⁶, 4×10⁶, 5×10⁶, 6×10⁶, 7×10⁶ 8×10⁶, 9×10⁶ 1×10⁷, 2×10⁷, 3×10⁷, 4×10⁷, 5×10⁷, 6×10⁷, 7×10⁷, 8×10⁷, 9×10⁷, 1×10⁸, or 2×10⁸ particles/mL.

In some embodiments, the microbubble composition of the present disclosure comprises at least one first medium and a plurality of microbubbles dispersed in the first medium.

In some embodiments, simulation of in vitro models before entering animal experiments indicates that when the dialysis membrane (such as hydrophilic cellulose membrane) is used instead of the tympanic membrane and the round window membrane, the dialysis membrane can increase the permeability of the dialysis membrane and drugs.

In some embodiments, the microbubble composition of the present disclosure is administrated to the specific cavity, such as middle ear cavity. In one embodiment, the microbubble composition can be mixed with the drug and applied into the middle ear cavity, and ultrasound can be applied to the ear canal or the skull behind the ear to enhance the effect of the inner ear on drug absorption. In one embodiment, the microbubble composition can be mixed with the drug and applied onto the round window membrane of the middle ear cavity, and ultrasound can be applied to the ear canal or the skull behind the ear to enhance the effect of the inner ear on drug absorption.

In theory, when the ultrasonic probe is in direct contact with the microbubble composition, the cavitation is induced by the ultrasonic energy. If the microbubble composition is mixed with the drug and applied to the middle ear cavity, the skull must be removed and the ultrasonic probe should be inserted into the middle ear cavity to contact the microbubble composition. In order to avoid infection and complications caused by excessively large wounds in the skull, if the hole is small, a slender ultrasonic probe must be used. However, because the limitation of current technology, the slender ultrasonic probe will not provide sufficient ultrasonic energy, so that the microbubble composition cannot produce a sufficient intensity of cavitation.

Therefore, some embodiments of the present disclosure provide transcanal and transcranial ultrasound-induced microbubble cavitation, and the microbubbles in the middle ear cavity are induced by the ultrasound probe applied in a non-invasive way to produce cavitation. Non-invasive treatment mode should be tried and tested in clinical application. Thus, the present disclosure provides transcanal and transcranial ultrasound-induced microbubble cavitation to achieve low-invasive inner ear treatment.

In some embodiments, the ultrasound probe includes a transducer.

In some embodiments, the choice of ultrasonic energy is based on inducing the cavitation. Therefore, the choices of ultrasonic energy in both animal experiment and clinical experiment are adjusted based on the principle of inducing the cavitation. In some embodiments, ultrasonic energy includes, but is not limited to 2 W/cm² to 10 W/cm². In some embodiments, ultrasonic energy includes 0.5 W/cm²-5 W/cm², 1 W/cm²-5 W/cm², 1 W/cm²-4 W/cm², or 1 W/cm²-3 W/cm². In some embodiments, ultrasonic energy includes 0.1 W/cm², 0.2 W/cm², 0.3 W/cm², 0.4 W/cm², 0.5 W/cm², 0.6 W/cm², 0.7 W/cm², 0.8 W/cm², 0.9 W/cm², 1 W/cm², 1.5 W/cm², 2 W/cm², 2.5 W/cm², 3 W/cm², 3.5 W/cm², 4 W/cm², 4.5 W/cm², 5 W/cm², 5.5 W/cm², 6 W/cm², 6.5 W/cm², 7 W/cm², 7.5 W/cm², 8 W/cm², 8.5 W/cm², 9 W/cm², 9.5 W/cm², or 10 W/cm².

In some embodiments, the difference in drug delivery between the experimental groups and the control group in which the drug was round window soaking in vivo was approximately 1.5 folds to 2.9 folds. In safety assessment, in some embodiments, it has been confirmed that techniques such as injecting microbubbles into the middle ear cavity by puncturing the eardrum with needle and applying the ultrasound, and transcanal or transcranial ultrasound-induced microbubble cavitation will not cause the complications of hearing impaired.

In some embodiments, the microbubble composition mixed with the drug is injected into the middle ear cavity, and transcanal or transcranial ultrasound-induced microbubble cavitation enhances the permeability of the round window membrane. The drug can penetrate into the cochlea through the round window membrane for safe and effective drug delivery.

### EXAMPLE

Although a series of operations or steps are used below to describe the method disclosed herein, an order of these operations or steps should not be construed as a limitation to the present invention.

### Example 1 the Preparation of Microbubble Ultrasound Contrast Agent

A total of 10 milliliters (mL) of saline (pH 7.4, 0.9% sodium chloride) and 140 milligrams (mg) of albumin were mixed uniformly, a perfluorocarbon gas (e.g. octafluoropropane, C₃F₈) was injected, and the microbubble ultrasound contrast agent was generated by 2 minutes sonication with a sonicater. The microbubble ultrasound contrast agent had a diameter of 1.02±0.11 μm and a concentration of 1.40×10⁸ MBs/mL, and the microbubbles were octafluoropropane coated with albumin as the spherical shell. Original concentration (1×) of microbubbles (MB1: 1.40×10⁸ MBs/mL) were prepared or 10-fold (10×) dilution of microbubbles (MB10: 1.40×10⁷ MBs/mL) were diluted with saline.

### Example 2 Measurement of Cavitation of Direct Ultrasound-Induced Microbubble with Different Concentrations

2% agarose square-column phantom (tissue-mimicking) (10×20×20 mm³) was constructed with a 2×2×20 mm³ chamber at its center, and 1×(MB1) or 10×(MB10) of the microbubble ultrasound contrast agent was injected. A sonoporation gene transfection system (ST 2000V, NepaGene, Ichikawa, Japan) having a 10 mm diameter transducer operating at a center frequency of 1 MHz was used to generate stable cavitation or an inertial cavitation at 3 W/cm² ultrasonic energy for directly giving to the microbubble ultrasound contrast agent (MB1 and MB10) in the chamber; and after covering the guinea pig's skull at the top of the chamber, the transducer also generated stable cavitation or an inertial cavitation at 3 W/cm² ultrasonic energy for transcranial administration to the in chamber with the microbubble ultrasound contrast agent (MB1+transcranial and MB10+transcranial). Before and after the ultrasound was applied, images of the microbubble ultrasound contrast agent was taken with a high-frequency ultrasound imaging system (US animal-imaging system, Prospect, S-Sharp Corporation, New Taipei City, Taiwan) with a center frequency of 40 MHz, and the image intensity of each group was calculated by Matlab program for calculating the area under the curve (AUC). Destruction efficiency was calculated by the following formula (I):

$$Destruction efficiency = \frac{brightness before ultrasound ( L 0 ) - brightness after ultrasound ( L n )}{brightness before ultrasound ( L 0 )} \times 100 \% \qquad formula ( I )$$

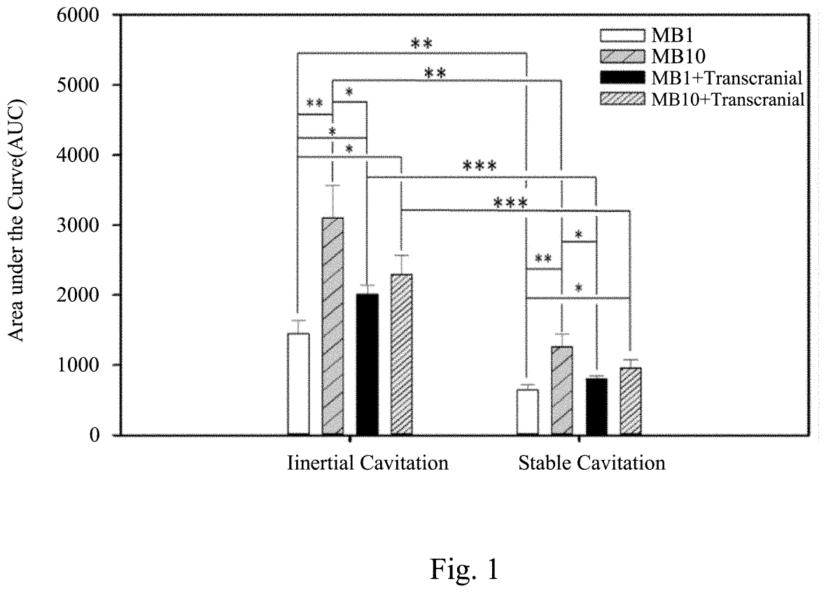

The results are shown in FIG. 1. In the inertial cavitation, the ultrasonic waves were direct administration or transcranial administration undiluted and 10-fold diluted microbubble ultrasound contrast agent. The area under the curve (AUC) was 1444.71±340.33, 3102.14±898.98, 2005.86±219.63, and 2292.2±554.86. In the stable cavitation, the ultrasonic waves were direct administration or transcranial administration undiluted and 10-fold diluted microbubble ultrasound contrast agent. The area under the curve (AUC) was 640.59±145.33, 1263.24±323.22, 796.16±87.59, and 959.77±241.98. From the above results, it is known that the inertial cavitation was stronger than the stable cavitation regardless of direct or transcranial administration at the same concentration of microbubble ultrasound contrast agent, and there was a significant difference (p<0.001). From the perspective of the inertial cavitation or stable cavitation, the signal strength of the microbubble ultrasound contrast agent diluted 10-fold was significantly stronger than that of the microbubble ultrasound contrast agent without dilution (p<0.01).

### Example 3 Measurement of Cavitation of Direct Ultrasound-Induced Microbubble

The embodiment is the same as that in Example 2, except that the inducer directly administrated the microbubble ultrasound contrast agent at 1 W/cm², 2 W/cm², 3 W/cm², and 4 W/cm² to generate inertia cavitation (e.g. with a duty cycle of 50% and the pulse repetition period is 250 milliseconds) of ultrasonic energy for 1 minute (n=5), and the temperature increase in the chamber was measured.

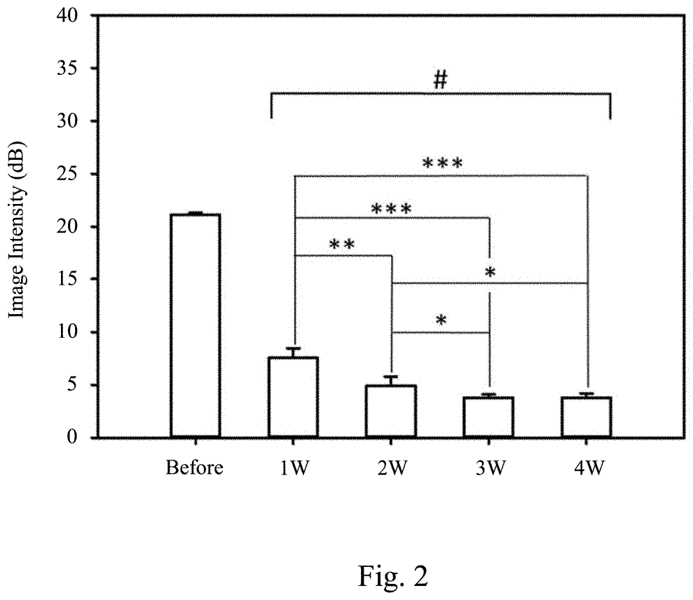

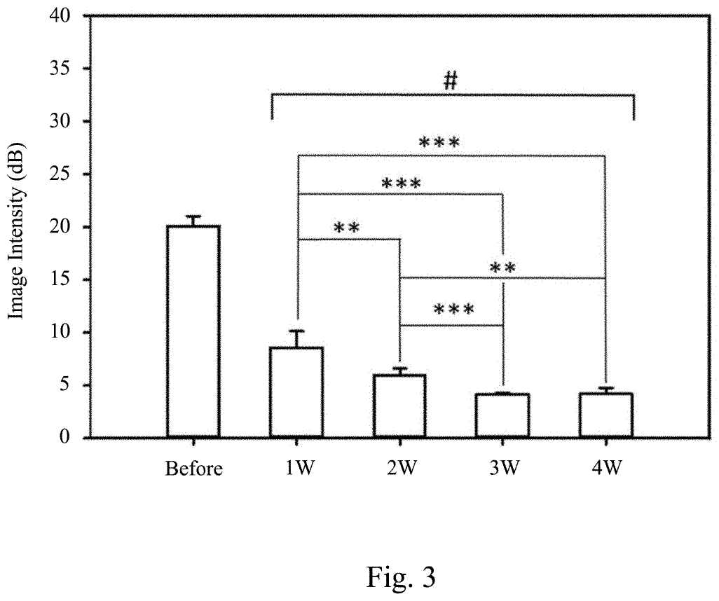

The results are shown in FIG. 2. The image intensities show that the intensity before applying ultrasounds was 21.1±0.2 dB, after the ultrasounds were applied continuously at 1 W/cm², 2 W/cm², 3 W/cm², and 4 W/cm², respectively for 1 minute, the image intensities were 7.54±0.93 dB, 4.91±0.86 dB, 3.78±0.31 dB, and 3.8±0.39 dB. The breaking efficiency using the formula (I) and the image intensities before ultrasonic administration to converse were 64.31%, 76.77%, 82.08%, and 82.02%, respectively, and all had significant differences (p<0.001). The above results show that when the ultrasonic energy was applied at 3 W/cm², the breaking efficiency was more than 80% in one minute, and the breaking efficiency was not significantly improved after increasing the ultrasonic energy to 4 W/cm². In addition, after applying ultrasonic energy at 3 W/cm², the temperature in the chamber changed in 1.75±0.15° C. without a significant increase.

### Example 4 Measurement of Cavitation of Transcranial Ultrasound-Induced Microbubble

The embodiment is the same as that in Example 2, except that the inducer transcranially administrated the microbubble ultrasound contrast agent at 1 W/cm², 2 W/cm², 3 W/cm², and 4 W/cm² to generate inertia cavitation of ultrasonic energy and extend for 3 minutes.

The above results show that when the ultrasonic energy was applied at 3 W/cm², the breaking efficiency was more than 79% after 3 minutes, and the breaking efficiency was not significantly improved after increasing the ultrasonic energy to 4 W/cm². In addition, after applying ultrasonic energy at 3 W/cm², the temperature in the chamber changed in 2.04±0.29° C. without a significant increase.

### Example 5 Measurement of Cavitation of Ultrasound-Induced Microbubble with Different Medium

The embodiment is the same as that in Example 2, except that the top of the chamber was covered with guinea pig's eardrum, and the ultrasonic energy of 3 W/cm² producing inertial cavitation was applied on the eardrum for 1 minute. At the same time, ultrasound was directly applied on the top of the chamber or was applied on the top of the chamber covered with the skull with the same energy and the same duration for comparison. The experimental groups were direct ultrasound administration (US), ultrasound through eardrum administration (US+ED), and ultrasound transcranial administration (US+B).

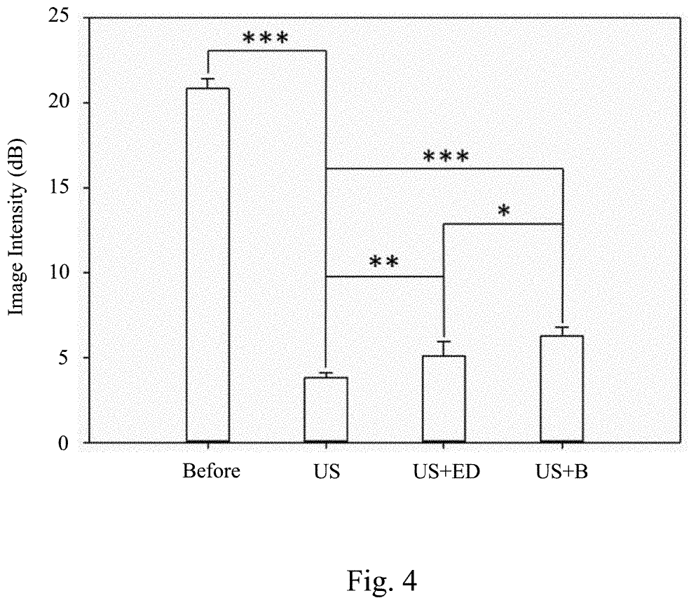

The quantification results are shown in FIG. 4, the image intensity before applying the ultrasound was 20.1±0.94 dB, and the image intensities after applying the ultrasonic energy of 3 W/cm² for 1 minute were 3.78±0.31 dB, 5.09±0.84 dB, and 6.26±0.52 dB. The above results show that direct ultrasound administration had about 80% of microbubble destruction efficiency and significant difference (p<0.001), ultrasound through the eardrum had about 75% of microbubble destruction efficiency and significant difference (p<0.01), and ultrasound transcranial administration had 68% microbubble destruction efficiency and significant difference (p<0.05). The experiment results indicate that ultrasound through eardrum could effectively destruct the microbubble ultrasound contrast agent, and confirm the feasibility of ultrasound through the eardrum to the middle ear cavity to promote drug delivery into the inner ear.

### Example 6 In Vitro Drug Delivery Model of Ultrasound Transcranial Administration with Microbubbles to Promote Drug Release to the Inner Ear

In order to understand the effect of ultrasound transcranial administration (behind the ear) to release the drug through the round window membrane (single-membrane), and the effect of ultrasound transcanal administration to release the drug through the eardrum and the round window membrane (two-membranes), 3D-printed material extrusion was used for designing three drug delivery devices:

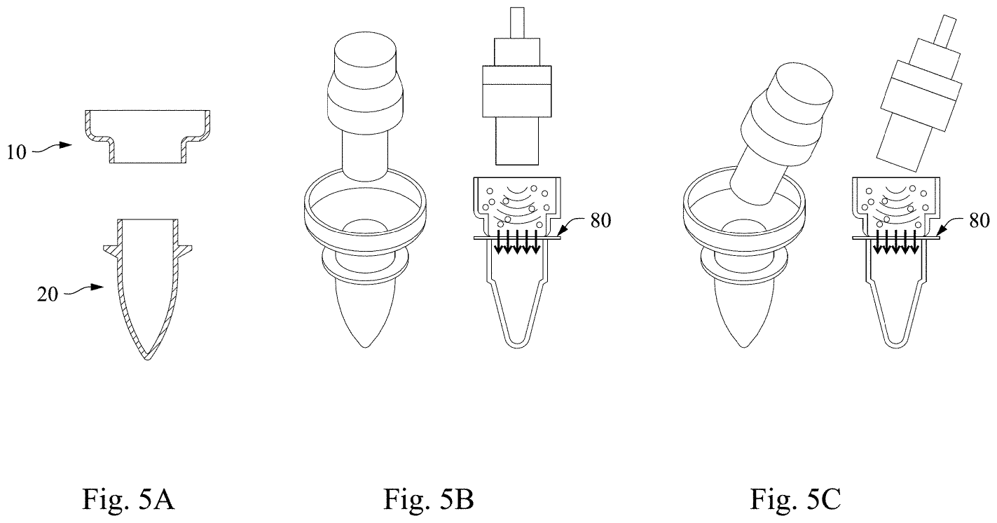

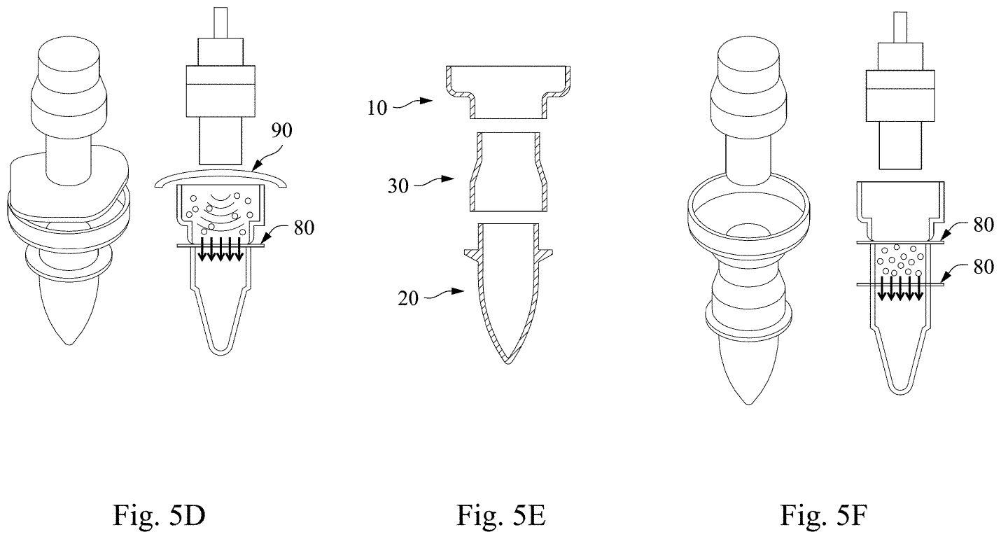

M1: single-membrane group (FIGS. 5A to 5C), a dialysis membrane 80 was sandwiched between the donor end 10 (Donor area) and the receiving end 20 (Receptor area) for mimicking the position of the round window membrane.

M1-20°: single-membrane with tilt group, the round window membrane of the guinea pig's ear was not perpendicular to the ear canal, so the ultrasonic probe originally perpendicular to the donor end 10 was tilted at an angle of about 20 degrees (FIG. 5C) to make the experiment more realistic. That is, after the top of the ultrasonic probe was placed at the surface center of saline in the donor end 10, the angle between the long axis direction of the ultrasonic probe and the long axis direction of the device was about 20 degrees.

M1+B: The single-membrane transcranial group (FIG. 5D) differed from the single-membrane group in that the skull 90 was placed on top of the donor end 10; and

M2: two-membranes group (FIGS. 5E to 5F), the dialysis membranes 80 were respectively sandwiched between the donor end 10 and the interval chamber 30 of the device, and between the interval chamber 30 and the receiving end 20, where the donor end 10 simulated the external ear canal, the interval chamber 30 simulated the middle ear cavity, and the receiving end 20 simulated the inner ear.

The above groups simulated the actual guinea pig's ear structure and the volume in each area. The printing material was polyethylene terephthalate (PETG). A suitable molecular weight (Orange scientific, OrDial D-Clean, MW=1000) was chosen for the dialysis membrane 80 to separate the sample from the tested drug. About 400 μL of saline was injected into the receiving end 20 to simulate the actual inner ear lymph fluid volume of the guinea pig. The experiment samples were diluted with microbubble ultrasound contrast agent 10-fold to 1000 μL, and mixed with 1 μL of biotin-FITC solution. The experiment samples were injected to the donor end 10 of the single-membrane group, the single-membrane transcranial group and the single-membrane with tilt group, and were injected to the interval chamber 30 of the two-membranes group, to mimic the volume of the middle ear cavity in the middle ear cavity of the guinea pigs. The single-membrane group and the two-membranes group were subjected to 3 W/cm² ultrasonic waves for 3 times (1 minute each time), while the single-membrane transcranial group blocked by the skull 90 was extended to 3 times (3 minutes each time) to ensure inducing the cavitation. The solution at the donor end 10 was replaced with a new one to ensure that the microbubble ultrasound contrast agent could produce a cavitation each time. In each group, saline in the receiving end 20 before the experiment, saline in the donor end 10 or the interval chamber 30 after the experiment, and saline in the receiving end 20 after the experiment were sampled for detecting fluorescence value.

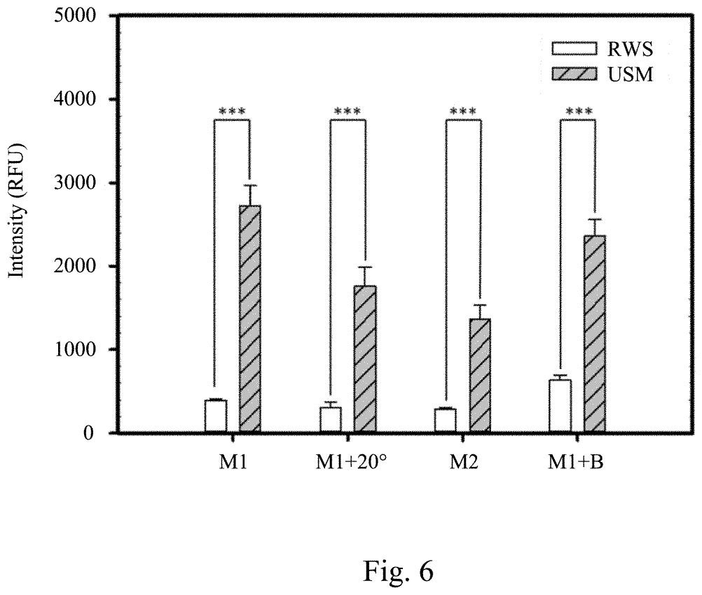

The results are shown in FIG. 6, the relative fluorescence units (RFU) of saline in the receiving end 20 of the single-membrane group (M1) with round window soaking (RWS) and ultrasound-induced microbubble cavitation treatments (USM) were 394.5±5.3 RFU and 2726.3±79.97 RFU respectively, and the ratio of permeability was about 6.91 folds with a significant difference (p<0.001). The relative fluorescence unit of the M1-20° group with RWS and USM were 309.7±20.54 RFU and 1756.3±76.29 RFU respectively, and the ratio of permeability was about 5.67 folds with a significant difference (p<0.001). The relative fluorescence unit of the M2 group with RWS and USM were 285.7±7.45 RFU and 1363.7±56.66 RFU respectively, and the ratio of permeability was about 4.77 folds with a significant difference (p<0.001). The relative fluorescence unit of the M1+B group with RWS and USM were 631.5±21.12 RFU and 2362±64.91 RFU respectively, and the ratio of permeability was about 3.74 folds with a significant difference (p<0.001). The results showed that the use of a two-membranes drug delivery model and a single-membrane transcranial drug delivery model to apply ultrasound were approximately 4.77 folds and 3.74 folds higher than the round window soaking. This indicates that even if the ultrasound probe was indirectly applied over a long distance, it could still effectively induce the cavitation by the microbubble ultrasound contrast agent, which further confirms the feasibility of transcanal and transcranial administration of ultrasound to promote drug delivery to the inner ear.

### Example 7 In Vivo Drug Penetration Analysis of Ultrasound Administration with Microbubbles Ultrasound Contrast Agent to the Inner Ear

Pigmented guinea pigs with normal sound reflection (Preyer's reflex) were used. The experimental group was divided into the following four groups:

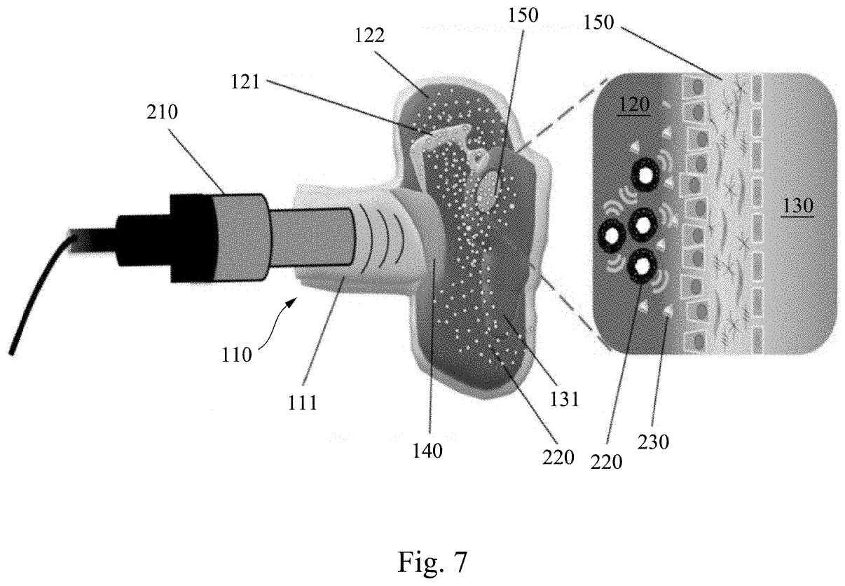

(1) Transcanal group (USM): the eardrum was punctured by a 22G needle and injected about 300 μL of a 10-fold diluted microbubble ultrasound contrast agent mixed with Biotin-FITC solution into the middle ear cavity. The external auditory canal was filled with saline as an ultrasound transmission medium, 3 W/cm² ultrasound (about 0.266 MPa) was applied to the external auditory canal 3 times (1 minute each time), and the solution in the middle ear cavity was replaced every time to ensure that the microbubble ultrasound contrast agent could produce a cavitation each time. FIG. 7 shows a schematic diagram of transcanal ultrasound-induced microbubble cavitation. The structure of the ear includes an outer ear 110, a middle ear 120, an inner ear 130, a tympanic membrane 140, and a round window membrane 150. The outer ear 110 includes an external ear canal 111, the middle ear 120 includes auditory ossicles 121 and a middle ear cavity 122, and the inner ear 130 includes a cochlea 131. The ultrasonic device 210 was placed in the external ear canal 111, and the ultrasound waves were generated by an ultrasonic device 210. The ultrasound waves were transmitted to the middle ear cavity 122 and induced the cavitation on the microbubbles 220 located in the middle ear cavity 122.

(2) RWS group A: the condition of the above-mentioned transcanal group was used, but the microbubble ultrasound contrast agent mixed with Biotin-FITC solution was stood for 3 minutes without applying ultrasounds.

(3) Transcranial group (USM): about 300 μL of 10-fold diluted microbubble ultrasound contrast agent mixed with Biotin-FITC solution was injected in to the middle ear cavity, and the skull skin behind the ear corresponding to the tympanic bulla was marked by a singular pen and applied with conductive glue. 3 W/cm² ultrasound was administered three times (3 minutes each time) at the mark place, and the solution in the middle ear cavity was replaced every time.

(4) RWS group B: the condition of the above-mentioned transcranial group was used, but the microbubble ultrasound contrast agent mixed with Biotin-FITC solution was stood for 9 minutes without applying ultrasounds.

After the animal experiment (n=4), the animals were sacrificed and the tympanic bullae were taken out. About 10 μL of perilymph fluid was collected and centrifuged. The supernatant was diluted at a ratio of 1:100 for detecting fluorescence value.

After the animal experiment had finished (n=4), the animals were sacrificed and the tympanic bullae were taken out. About 10 μL of perilymph fluid was collected and centrifuged. The supernatant was diluted at a ratio of 1:100 for detecting fluorescence value.

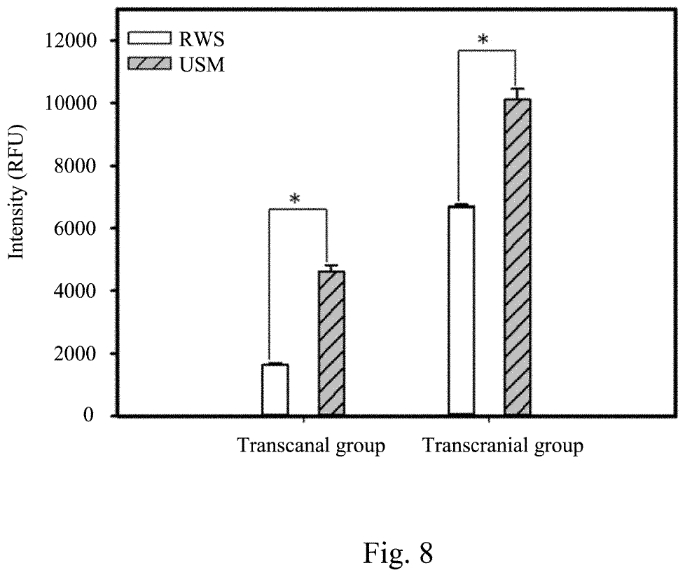

The results are shown in FIG. 8, the relative fluorescence units of RWS group A and the transcanal group (USM) were 1633.33±72.63 RFU and 4206.83±202.93 RFU respectively, and the ratio of permeability was about 2.83 folds with a significant difference (p<0.05). The relative fluorescence units of RWS group B and transcranial group (USM) were 6700±69.74 RFU and 10113±338.8 RFU respectively, and the ratio of permeability was about 1.5 folds with a significant difference (p<0.05). Animal experiments have proven that the transcanal and transcranial administration had excellent permeability.

### Example 8 Auditory Brainstem Response Examination

In order to detect the effect of ultrasound transcanal administration with microbubbles ultrasound contrast agent on guinea pigs' hearing (n=4), hearing was detected at the lowest decibel level being audible (hearing threshold) on the 14th and 28th days after the animal experiment in Example 7.

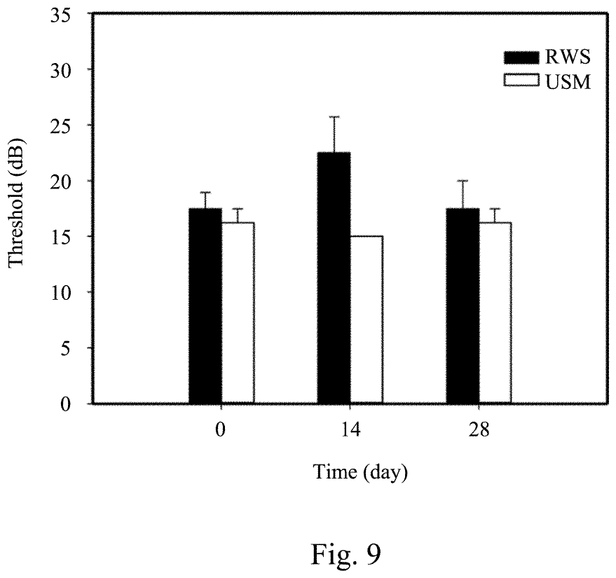

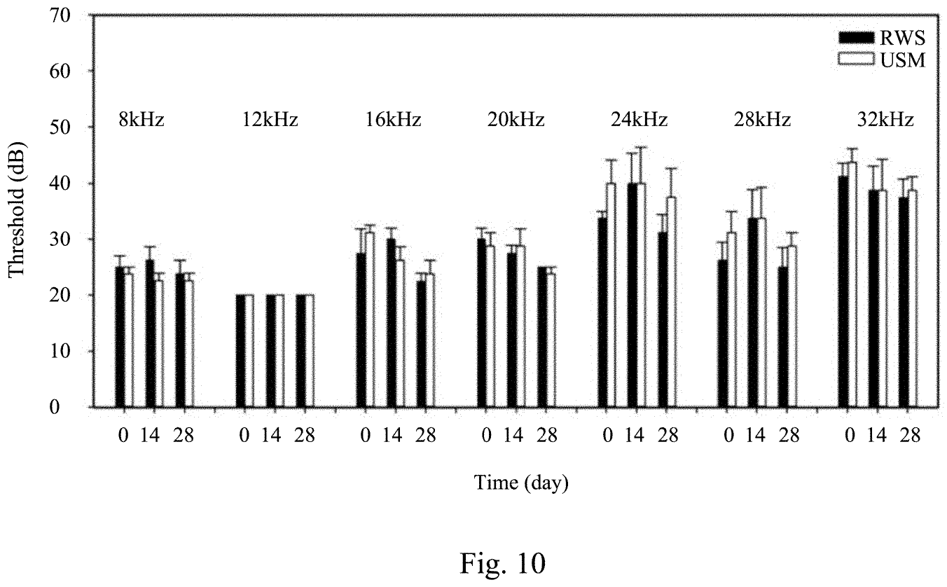

The results were shown in FIGS. 9 and 10, the normal hearing of guinea pigs before surgery (0 day) at full frequency was 15-20 decibels (dB) (FIG. 9), and the normal hearing at frequencies of 8K-32K (Hz) were approximately at around 20-40 dB (FIG. 10). On the 14th day after the animal experiment of Example 7, the lowest decibel of full frequency that could be heard (hearing threshold) was 15-25 decibels (FIG. 9) and the frequencies of 8K-32K (Hz) were about 20-45 dB (FIG. 10), both were slightly higher than that of the decibels before surgery (0 day). On the 28th day after the surgery, the results show that the hearing recovered to the preoperative level. That is, puncturing the eardrum and injecting microbubble ultrasound contrast agent with applying ultrasound will cause a short-term hearing loss, but the hearing performance can still recover after about 1 month to preoperative standards.

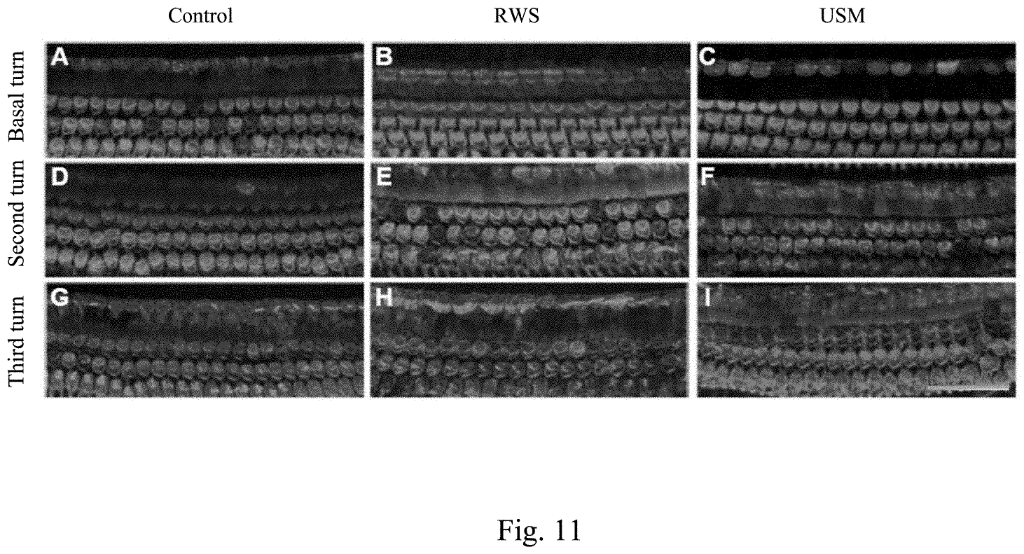

In addition, the results are shown in FIG. 11 that the left column showed the situation in which the cochlear hair cells under normal conditions (control group), the middle column showed RWS, and the right column showed the USM. After comparing USM and RWS, it can be found that the hair cells were aligned at the basal turn, second turn, and third turn of the cochlea structure after transcanal administration, and the hair cells were not lost. Therefore, it can be seen that ultrasound transcanal administration does not affect cochlear hair cells.

### Example 9

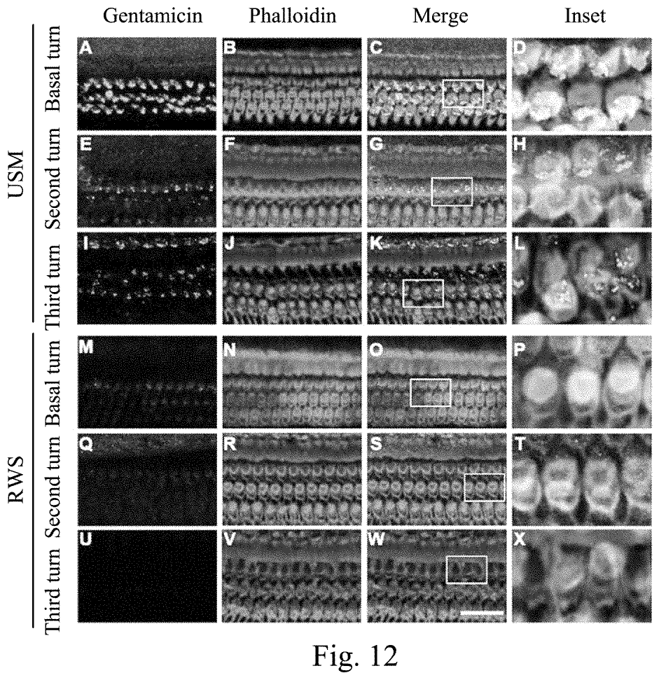

Gentamicin is an antibiotic of ototoxic drugs, so it is easily swallowed by the inner ear to mimic the position where the drug can be delivered. The animal experiment surgery procedure of gentamicin combined with 10-fold diluted microbubble ultrasound contrast agent was similar to that of the transcanal group in Example 7. Round-window soaking group was similar to the condition of the above-mentioned group, but the microbubble ultrasound contrast agent mixed with gentamicin was stood for 3 minutes without applying ultrasounds. Because phalloidin binds to the cytoskeletal actin, it is used to mark the location of hair cells. In the animal experiment results of gentamicin administration, the far left column in FIG. 12 showed the distribution of gentamicin in the cochlear hair cells, and the second column of the phalloidin indicated the location of the hair cells. Merge column is the overlay of the left two columns, it is more obvious to see the distribution of gentamicin in the cochlea. After comparing the upper and lower graphs of RWS and USM, it can be confirmed whether the ultrasound-microbubble administration significantly affected the delivery of the drug. The green dots indicated the uptake of gentamicin in the hair cells. In the RWS figures, only a small amount of green dot-like fluorescence could be found in the basal turn, the second turn and third turn did not found the dot-like fluorescence. In the USM figures, green fluorescence was found in the basal turn, the second turn, and third turn. In the cochlea structure, the drug had significant permeability because the basal turn was the closest region to the round window membrane. Therefore, the image of ultrasound transcanal administration with microbubble ultrasound contrast agent is significantly different from that of the RWS.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of delivering drugs to inner ear facilitated by microbubbles, comprising:
providing a microbubble composition, wherein the microbubble composition comprises at least one first medium and a plurality of microbubbles dispersed in the first medium;
providing a drug;
mixing the microbubble composition with the drug to form a microbubble-drug mixture;
applying the microbubble-drug mixture to the middle ear cavity; and
applying a mechanical oscillation wave source to be non-invasive and indirect contact with the microbubble-drug mixture, wherein mechanical waves are generated by the mechanical oscillation wave source, the microbubbles of the microbubble-drug mixture in the middle ear producing a cavitation is induced by the mechanical waves, so as to increase the permeability of the round window membrane, thereby allowing the drug in the microbubble-drug mixture to penetrate the round window membrane into the inner ear.

2. The method of claim 1, wherein after the step of applying the microbubble-drug mixture to the middle ear cavity, the method further comprises filling a second medium into an ear canal;
wherein the step of applying a mechanical oscillation wave source to be non-invasive and indirect contact with the microbubble-drug mixture, the mechanical oscillation wave source is in contact with the second medium in the ear canal, and the mechanical waves penetrate the eardrum and the microbubbles in the microbubble-drug mixture located in the middle ear are induced to produce the cavitation.

3. The method of claim 2, wherein the second medium comprises saline, a gel, or a combination thereof.

4. The method of claim 1, wherein the step of applying the mechanical oscillation wave source to be non-invasive and indirect contact with the microbubble-drug mixture, the mechanical oscillation wave source placed at a skull near an ear shell generates mechanical waves without chiseling the skull, and the mechanical waves penetrate the skull and the microbubbles in the microbubble-drug mixture located in the middle ear are induced to produce the cavitation.

5. The method of claim 1, wherein the cavitation is a stable cavitation or an inertial cavitation.

6. The method of claim 1, wherein the step of applying the microbubble-drug mixture to the middle ear cavity is to apply the microbubble-drug mixture to a round window membrane toward a middle ear cavity.

7. The method of claim 1, wherein the first medium comprises saline, a gel, or a combination thereof.

8. The method of claim 1, wherein the material of the microbubbles comprises albumin, a lysozyme, a polymer, a liposome or a combination thereof.

9. The method of claim 1, wherein the microbubbles have a particle size from 0.5 μm to 2.5 μm.

10. The method of claim 1, wherein the mechanical oscillation wave source comprises an ultrasonic device, a laser device, or a combination thereof.

11. The method of claim 1, wherein the concentration of the microbubbles in the microbubble-drug mixture ranges from 1×10⁶ to 2×10⁸ particles/m L.
